(19) United States
(12) Patent Application Publication
(10) Pub. No.: US 2010/0215663 A1
(43) Pub. Date: Aug. 26, 2010

(54) METHODS AND COMPOSITIONS FOR REGULATION OF NEUROLOGICAL CONDITIONS

(75) Inventors: Jaime Grutzendler, Chicago, IL (US); Zhiqiang Liu, Chicago, IL (US)

Correspondence Address: Casimir Jones, S.C., 2275 DEMING WAY, SUITE 310, MIDDLETON, WI, 53562, US

(73) Assignee: Northwestern University, Evanston, IL (US)

(21) Appl. No.: 12/279,499

(22) PCT Filed: Feb. 15, 2007
(86) PCT No.: PCT/US2007/004097

§ 371 Date: Aug. 14, 2008

Related U.S. Application Data
(60) Provisional application No. 60/773,705, filed on Feb. 15, 2006.

(51) Int. Cl. A61K 39/395; A61K 38/16; A61P 25/28; G01N 33/68

(52) U.S. Cl. 424/158.1; 514/12; 436/86

(57) ABSTRACT

The invention relates to methods and compositions for regulation of neurological conditions. In particular, methods and compositions for the modulation of the chemokine receptor 1 (CX3CR1) and its ligands are described.

### FIELD OF THE INVENTION

The invention relates to methods and compositions for regulation of neurological conditions. In particular, methods and compositions for the modulation of the chemokine receptor 1 (CX3CR1) and its ligands are described.

### BACKGROUND OF THE INVENTION

Neurological conditions, such as Parkinson's Disease, Amyotrophic Lateral Sclerosis (ALS or Lou Gehrig's Disease), and Alzheimer's Disease are progressive disorders of the central nervous system that affect millions of people in the United States alone. These neurological disorders are tremendously debilitating to all those affected, and there are no known cures or treatments that significantly offset the severity of these disorders. Parkinson's Disease is caused by the degeneration of neurons in the brain. To date, both medication and surgical treatment options are available to treat the disease, but unfortunately all of the available medications can cause undesirable side effects. Surgical intervention to treat Parkinson's Disease is even more risky, with the possibility of stroke being a potential outcome. ALS is caused by the progressive degeneration of motor neurons leading to muscle weakness, atrophy, eventual total body paralysis, and death. Most people afflicted with ALS will, however, remain cogent to the very end as mental functions are typically unaffected. Alzheimer's Disease is the most common cause of dementia, and leads to nerve cell death and tissue loss throughout the brain, affecting nearly all its functions. As with Parkinson's Disease, there is no cure for ALS or Alzheimer's Disease. There are medications that help some Alzheimer's afflicted patients by improving their cognitive symptoms, however, as with Parkinson's Disease, the medications and current treatments affect patients differentially. There are no known treatments for ALS. What is needed are novel ways of understanding, studying, and treating these types of debilitating, neurological disorders.

### SUMMARY OF THE INVENTION

The present application describes novel methods and compositions for modulating neurological disorders, such as neuroinflammatory and neurodegenerative diseases, by providing methods and compositions for the modulation of the CX3C chemokine receptor 1 (CX3CR1) and its ligands (e.g. fractalkine). The study of this chemokine receptor and its ligands, and their association with neurological disorders, also serve as a useful tool for understanding the science surrounding these disorders. Neuroinflammation is generally characterized by microglia proliferation, and activation as well as infiltration of the brain by peripheral macrophages, lymphocytes and neuotrophils, which can subsequently lead to neurodegeneration. Neurological disorders associated with neuroinflammation and neurodegeneration include, but are not limited to Alexander disease (Hanefield, F, 2004, Cell. Mol. Life Sci. 61:2750-2), Alper's disease (Prick, M J et al, 1982, Neuroped. 13:108-11), Alzheimer's disease (Gottfires C G, 1988, Comp. Gerentol. 2:47-62), Amytrophic lateral sclerosis (Hudson A J, 1981, Brain 104:217-47), Louis-Bar syndrome (Boder R et al, 1958, Pediatrics 2:526-54), Canavan disease (Surendran S et al, 2003, Mol. Genet. Metab. 80:74-80), Cockayne syndrome (van Gool A J et al, 1997, EMBO J. 16:4155-62), corticobasal degeneration (Gibb W R et al, 1989, Brain 112:1171-92), Creutzfeldt-Jakob disease (Johnson R T et al, 1998, N. Engl. J. Med. 339:1994-2004), Huntington disease (Myrianthopoulos N C, 1966, J. Med. Genet. 3:298-314), Kennedy's disease (Kennedy W R et al, 1968, Neuro. 18:671-80), Krabbe disease (Wenger D A et al, 1997, Hum. Mutat. 10:268-79), Lewy body dementia (Zaccai J et al, 2005, Age Ageing. 34:561-6), Machado-Joseph disease (Sudarsky L et al, 1995, Clin. Neurosci. 3:17-22), multiple sclerosis (Calabresi P A, 2004, Am. Fam. Physician 70:1935-44), multiple system atrophy (Shy G M et al, 1960, Arch. Nuerol. 2:511-27), Parkinson's disease (Masliah E et al, 2000, Science 287:1265-9), Pelizaeus-Merzbacher disease (Uhlenberg B et al, 2004, Am. J. Hum. Genet. 75:251-60), Pick's disease (Neary D et al, 1998, Neuro. 51:1546-54), Refsum disease (Wierzbicki A S et al, 2002, J. Neurochem. 80:727-35), Sandhoff disease (Hendriksz C J et al, 2004 J. Inherit. Metab. Dis. 27:241-9), Schilder's disease (Kotil K et al, 2002, Br. J. Neurosurg. 16:516-9), spinal muscular atrophy (Ogino S et al, 2004 Expert. Rev. Mol. Diagn. 4:15-29), Steele-Richardson-Olszewski disease (Steele J C et al, 1964, Arch. Neurol. 10:333-59), and tabes dorsalis (Nitrini R, 2000, Arch. Neurol. 57:605-6).

Chemokines are a family of molecules that are released by the body's tissues, such as brain tissue, and one of their functions is to recruit effector cells, such as neutrophils and other white blood cells, to sites of injury and/or inflammation. The continuous release of chemokines at sites of inflammation can mediate the continuous migration and recruitment of effector cells to chronic inflammation sites. The present invention describes methods and compositions surrounding the activities of a particular class of chemokines known as CX3C chemokines, an example of which is the chemokine fractalkine, and the receptor that recognizes and binds to CX3C chemokines, known as the CX3C receptor 1 (CX3CR1). This ongoing recruitment of leukocytes to the brain and other central nervous system locations caused by chemokine signaling perpetuates neuroinflammation and can lead to neurodegeneration and neuronal death, thereby contributing to the symptoms and ongoing disease states of, for example, Parkinson's Disease, ALS, and Alzheimer's Disease.

The present application demonstrates that by inhibiting the activity of CX3CR1 and/or its CX3C chemokine fractalkine, one can inhibit cellular responses (e.g., activation, migration, adhesion) mediated by the receptor and/or chemokine thereby inhibiting the initiation, progression and/or maintenance of neuroinflammatory disease and neuronal death.

In one aspect, the invention relates to methods and compositions for treating a subject having a neuroinflammatory disease. Treatments include therapeutic or prophylactic treatment that prevent the disease or reduce the severity of disease in whole or in part. It is contemplated that a method of treating a neuroinflammatory disease comprises administering an effective amount of an agent that blocks the activity of the CX3 CR1 and/or its ligands (e.g. fractalkine) to a subject in need thereof. In another aspect, the invention relates to methods and compositions for identifying substances that affect the activity of the CX3CR1 and its ligands, thereby inhibiting the initiation, progression and/or maintenance of neuroinflammatory disease and neuronal death.

Accordingly, the present invention provides assays (methods) and materials (compositions) suitable for the identification and evaluation of pharmacological agents that are potent inhibitors of activity of CX3CR1 and its ligands. It also provides assays and materials to identify and evaluate pharmacological agents that preferentially or selectively inhibit CX3CR1 and/or its ligands.

### DESCRIPTION OF THE FIGURES

FIG. 1 Shows the affect of CX3CR1 on dopaminergic neuron degeneration in SNpc of C57B16 mice in reponse to MPTP injury. Left panel shows the differential tyrosine hydroxylase immunoreactivities in three different CX3CR1 genotypes (A-CX3CR1 −/−; B-CX3CR1 +/−, C-CX3CR1 +/+) at day 28 after four intraperitoneal injection of MPTP. Right panel displays the quantification of dopaminergic neurons in SNpc (n=9, mean±SD, x-axis=neuron number, y-axis=mouse CX3CR1 genotype).

FIG. 2a & b shows the affect of a CX3CR1 knock-out genotype in a transgenic Alzheimer's Disease mouse model. A-sagittal section of mouse brain; arrows denote Alzheimer related amyloid plaques; B-Coronal section of mouse brain; light spots indicate Alzheimer related amyloid plaques, comparison of homozygous vs. heterozygous CX3CR1 genotyped cross-bred mice and Alzheimer related amyloid plaque number (y=number of amyloid plaques).

### DEFINITIONS

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include tissues and blood products, such as plasma, serum and the like. Such examples are not however to be construed as limiting the sample types applicable to the present invention.

As used herein, the term “peptide” refers to a compound comprising from two or more amino acid residues wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g. solid phase synthesis) or molecular biology techniques (see Sambrook, J. et al., Molecular Cloning: A Laboratory Manual, Cold Spring Harbor Press, Cold Spring Harbor, N.Y. (1989)).

As used herein, the term “peptidomimetic” refers to molecules which are not polypeptides, but which mimic aspects of their structures. For example, polysaccharides can be prepared that have the same functional groups as peptides. A peptidomimetic comprises at least two components, the binding moiety or moieties, and the backbone or supporting structure.

As used herein, the term “antibody” encompasses both monoclonal and polyclonal full length antibodies and functional fragments thereof (e.g. maintenance of binding to target molecule). Antibodies can include those that are chimeric, humanized, primatized, veneered or single chain antibodies.

As used herein, the term “neuroinflammatory disease” refers to those diseases of the nervous system where the immune system causes or exacerbates inflammation in the nerve tissue.

As used herein, the term “effective amount” of a therapeutic compound (e.g. agent, compound, or drug) is an amount sufficient to achieve a desired therapeutic and/or prophylactic effect, such as an amount sufficient to inhibit plaque formation, to inhibit neuronal cell death, or to alleviate behavioral disorders associated with a neuroinflammatory disease.

As used herein, the terms “agent”, “compound” or “drug” are used to denote a compound or mixture of chemical compounds, a biological macromolecule such as an antibody, a nucleic acid, or an extract made from biological materials such as bacteria, plants, fungi, or animal (particularly mammalian) cells or tissues that are suspected of having therapeutic properties. The compound, agent or drug may be purified, substantially purified or partially purified.

As used herein, the term “fragment” when in reference to a protein (e.g. “a fragment of a given protein”) refers to portions of that protein. The fragments may range in size from two amino acid residues to the entire amino acid sequence minus one amino acid. In one embodiment, the present invention contemplates “functional fragments” of a protein. Such fragments are “functional” if they can bind with their intended target protein (e.g. the functional fragment may lack the activity of the full length protein, but binding between the functional fragment and the target protein is maintained).

As used herein, the term “antagonist” refers to molecules or compounds (either native or synthetic) that inhibit the action of a compound. Antagonists may or may not be homologous to these compounds in respect to conformation, charge or other characteristics. Thus, antagonists may be recognized by the same or different receptors that are recognized by an agonist. Antagonists may have allosteric effects that prevent the action of an agonist. Or, antagonists may prevent the function of the agonist.

As used herein, the term “therapeutically effective amount” refers to that amount of a composition that results in amelioration of symptoms or a prolongation of survival in a patient. A therapeutically relevant amount relieves to some extent one or more symptoms of a disease or condition, or, returns to normal, either partially or completely, one or more physiological or biochemical parameters associated with a disease or condition.

### DETAILED DESCRIPTION OF THE INVENTION

The invention relates to methods and compositions for regulation of neurological conditions. In particular, methods and compositions for the modulation of the chemokine receptor 1 (CX3CR1) and its ligands are described.

Chemokines are a family of proinflammatory mediators that promote recruitment and activation of multiple lineages of leukocytes (e.g., lymphocytes, macrophages) and are released by many kinds of tissue cells after activation. Continuous release of chemokines at sites of inflammation can mediate the ongoing migration and recruitment of effector cells to sites of chronic inflammation. Chemokines are related in primary structure and share four conserved cysteines, which form disulfide bonds. Based upon this conserved cysteine motif, the family can be divided into distinct branches, including the C-C chemokines (β-chemokines), C-X-C chemokines (α.-chemokines), and the C-XXX-C chemokines (CX3C chemokines), in which the first two conserved cysteines are adjacent or separated by one or three intervening residues, respectively (see e.g., Baggiolini, M. and Dahinden, C. A., Immunology Today, 15:127-133 (1994); Bazan, J. F. et al., Nature, 385:640-644 (1997)). The CX3C chemokines include fractalkine (fkn), which is also referred to as neurotactin (Pan, Y. et al., Nature, 387:611-617 (1997)), CX3CL1 , CXXXCL1, ABCD-3 (Schaniel C., et al., Eur. J Immunol., 29:2934-2947 (1999)) and SCYDI (Nomiyama H. et al., Cytogenet. Cell Genet., 81:10-11 (1998)).

Fractalkine (fkn) is a transmembrane molecule that has an extra-cellular region containing a conserved chemokine domain atop a mucin-like stalk (Imai, T. et al., Cell, 91:521 (1997)). A soluble form of fractalkine, which may be the product of processing (e.g., proteolytic cleavage) of the transmembrane molecule, is produced by cells in vivo and in vitro. Fractalkine can function as a cellular adhesion molecule and as a chemoattractant for monocytes and lymphocytes (Imai, T. et al., Cell, 91:521 (1997); Kanazawa, N. et al., Eur. J. Immunol., 29:1925 (1999); Fong, A. et al., J. Exp. Med., 188:1413 (1998); Bazan, J. et al., Nature, 385:640 (1997)). The human CX3C chemokine receptor 1 (CX3CR1; Raport C J et al., 1995, Gene 163:295-299; WO 94/12635; Godiska et al, U.S. Pat. No. 5,759,804; incorporated herein in their entirety) can bind fractalkine and is expressed by a variety of different cells and tissues including peripheral blood leukocytes (PBL), spleen and brain.

Agents that inhibit the activity of CX3CR1 and/or fractalkine can inhibit cellular responses (e.g., activation, migration, adhesion) mediated by the receptor and/or chemokine and can inhibit the initiation, progression and/or maintenance of neuroinflammatory disease and neuronal death.

One embodiment of the present invention relates to therapeutic methods for treating a subject having neuroinflammatory disease. In some embodiments, the method of treatment comprises the administration of an antagonist, agent, compound, or drug to a subject having a neuroinflammatory disease. For example, a therapeutic method of treatment comprises the administration of an antagonist, such as an antibody or nucleic acid, to the CX3CR1 receptor of a subject having a neuroinflammatory disease. In some embodiments, the antagonist physically interacts with the receptor, or the antagonist blocks production of the receptor, e.g. by inhibiting translation of the receptor gene into a protein product. Another embodiment of a therapeutic method of treatment comprises the administration of an antagonist, such as an antibody or a nucleic acid, of a CX3CR1 ligand, such as fractalkine, to a subject having a neuroinflammatory disease. For example, in some embodiments the antagonist is an antibody that physically interacts with the ligand, or a nucleic acid that is responsible for blocking expression of the ligand, e.g. by inhibiting translation of the ligand gene mRNA.

In some embodiments the present invention provides methods of storage and administration of the antagonist, agent, compound, or drug in a suitable environment (e.g. buffer system, adjuvants, etc.) in order to maintain the efficacy and potency of the agent, compound, or drug such that its usefulness in a method of treatment of a neuroinflammatory disease is maximized. For example, protein agents, compounds or drugs benefit from a storage environment free of proteinases and other enzymes or compounds that could cause degradation of the protein.

A preferred embodiment is contemplated where the antagonist, agent, compound, or drug is administered to the individual as part of a pharmaceutical or physiological composition for treating neuroinflammatory disease. Such a composition can comprise an antagonist and a physiologically acceptable carrier. Pharmaceutical compositions for co-therapy can further comprise one or more additional therapeutic agents. Alternatively, an antagonist (e.g., an antagonist of CX3CR1 function and/or an antagonist of fractalkine function, as described herein) and an additional therapeutic agent are components of separate pharmaceutical compositions that are mixed together prior to administration, or administered separately. The formulation of a pharmaceutical composition can vary according to the route of administration selected (e.g., solution, emulsion, capsule). Suitable pharmaceutical carriers can contain inert ingredients that do not interact with the antagonist of CX3CR1 function and/or additional therapeutic agent. Standard pharmaceutical formulation techniques can be employed, such as those described in Remington's Pharmaceutical Sciences, Mack Publishing Company, Easton, Pa. Suitable physiological carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al, “Controlled Release of Biological Active Agents”, John Wiley and Sons, 1986). Therapeutic agents suitable for co-administration with an antagonist of CX3CR1 function and/or an antagonist of fractalkine function include, for example, methotrexate, anti-inflammatory agents (e.g., nonsteroidal anti-inflammatory agents, such as aspirin, ibuprofen, naproxen, lysofylline, inhibitors of cyclooxygenase-2), cytokines (e.g., TGFβ), immunosuppressive agents, such as, calcineurin inhibitors (e.g., cyclosporin A, FK-506), IL-2 signal transduction inhibitors (e.g., rapamycin), glucocorticoids (e.g., prednisone, dexamethasone, methylprednisolone), nucleic acid synthesis inhibitors (e.g., azathioprine, mercaptopurine, mycophenolic acid), and antibodies to lymphocytes and antigen-binding fragments thereof (e.g., OKT3, anti-IL2 receptor), disease modifying anti-rheumatic agents (e.g., D-penicillamine, sulfasalazine, chloroquine, hydroxychloroquine) and antibodies, such as antibodies that bind chemokines, cytokines (e.g., anti-TNFα) or cell adhesion molecules (e.g., anti-CD 11/CD 18). The particular co-therapeutic agent selected for administration with an antagonist of CX3CR1 and/or an antagonist of fractalkine function will depend on the type and severity of neuroinflarnmatory disease being treated as well as the characteristics of the individual, such as general health, age, sex, body weight and tolerance to drugs. Typically, an effective amount can range from about 0.01 mg per day to about 100 mg per day for an adult. Preferably, the dosage ranges from about 1 mg per day to about 100 mg per day. The skilled artisan will be able to determine the preferred co-therapeutic agent based upon these considerations and other factors.

In some embodiments the therapeutic agent is administered by any suitable route, including, for example, orally (e.g., in capsules, suspensions or tablets) or by parenteral administration. Parenteral administration can include, for example, intramuscular, intravenous, intraarticular, intrathecal, subcutaneous, or intraperitoneal administration. The therapeutic agent (e.g., CX3CR1 antagonist, fractalkine antagonist, additional therapeutic agent) can also be administered transdermally, topically, by inhalation (e.g., intrabronchial, intranasal, oral inhalation or intranasal drops) or rectally. Administration can be local or systemic as indicated. The preferred mode of administration can vary depending upon the particular agent (e.g., CX3CR1 antagonist, fractalkine antagonist, additional therapeutic agent) chosen, however, oral or parenteral administration is generally preferred. In some circumstances where high brain levels of the antagonist are desired, intrathecal injection or direct administration into the brain tissue is contemplated.

An additional embodiment for administration of a therapeutic agent (e.g., CX3CR1 antagonist, fractalkine antagonist, additional therapeutic agent) is administration of the therapeutic agent as a neutral compound or as a salt.

When co-administration of an antagonistic therapeutic agent (e.g., an antagonist of CX3CR1 function and/or an antagonist of fractalkine function, as described herein) and an additional therapeutic agent is indicated or desired for treating a neuroinflammatory disease, the antagonistic therapeutic agent (e.g., antagonist of CX3CR1 function and/or antagonist of fractalkine function) can be administered prior to, concurrently with, or subsequent to administration of the additional therapeutic agent. When the antagonistic therapeutic agent and the additional therapeutic agent are administered at different times, they are preferably administered within a suitable time period to provide substantial overlap of the pharmacological activity of the agents. The skilled artisan will be able to determine the appropriate timing for co-administration of antagonistic therapeutic agents (e.g., antagonist of CX3CR1 function and/or antagonist of fractalkine function) and an additional therapeutic agent.

The present invention provides methods for identifying antagonists, agents, drugs and compounds that are utilized to treat a subject with a neuroinflammatory disease. In some embodiments, the present invention provides drug-screening assays (e.g., to screen for drugs effective in treating neuroinflammatory diseases). For example, the present invention contemplates methods of screening for compounds that alter (e.g., increase or decrease) the expression level or activity of a CX3CR1 ligand. In one embodiment, the amount of a CX3CR1 ligand is detected in a subject that has undergone administration of a candidate compound. The increased amount of a CX3CR1 ligand is indicative of a candidate compound that is not preventing neuroinflammatory disease. In other embodiments, the present invention contemplates methods of screening compounds that alter (e.g., increase or decrease) the activity of CX3CR1 (e.g. binding activity of the CX3CR1). In some embodiments, the activity of CX3CR1 is detected in a subject that has undergone administration of a candidate compound. In some embodiments, the activity of CX3CR1 is detected using an in vitro assay, for example, an enzyme-linked immunosorbent assay, or other assays which utilize a labeled (e.g., fluorescent, luminescent, colorimetric, radioactive) compound for detection of receptor activity. Antagonists of CX3CR1 function can be identified, for example, by screening libraries or collections of molecules, such as the Chemical Repository of the National Cancer Institute, as described herein or using other suitable methods. Antagonists thus identified find use in the therapeutic methods described herein. Another source for identifying potential antagonists of CX3CR1 function are combinatorial libraries, which can comprise many structurally distinct molecular species. Combinatorial libraries can be used to identify compounds or to optimize a previously identified compound. Such libraries can be manufactured by well-known methods of combinatorial chemistry and can be screened by suitable methods, such as those described in Molecular Cloning: A Laboratory Manual Sambrook J et al Eds, Cold Harbor Spring Laboratory Press.

In some embodiments, drug screening assays are performed in animals. Any suitable animal may be used including, but not limited to, baboons, rhesus or other monkeys, mice, or rats. Animal models of neuroinflammatory disease are generated, and the effects of candidate drugs on the animals are measured. In preferred embodiments, neuroinflammatory diseases in the animals are measured by detecting levels of a CX3CR1 ligand in the affected tissues (e.g. brain, cerebrospinal fluid, other neuronal tissues) of the animals. The level of related CX3CR1 ligand may be detected using any suitable method, including, but not limited to, those disclosed herein.

The present invention is not limited by the nature of the antagonist used in the therapeutic or screening methods of the invention. In some embodiments, the CX3CR1 antagonist is an antibody or antigen-binding fragment thereof having specificity for CX3CR1. The antibody can be polyclonal or monoclonal. The antibody or antigen-binding fragment preferably has specificity for a mammalian CX3CR1, such as a human CX3CR1. In a preferred embodiment, the antibody or antigen-binding fragment inhibits binding of a ligand (e.g., one or more ligands) to CX3CR1 and/or one or more functions mediated by CX3CR1 in response to ligand binding. For example, agents, drugs and compounds that may be may be used in the methods of the invention include, but are not limited to, those found in the following references, incorporated by reference herein in their entirety; Chen S et al, 2002, J. Neuroimmunol. 133:46-55; Davalos D J et al, 2005, Nat. Neurosci. 8:752-8; Feng Let al, 1999, Kidney Int. 56:612-20; Fong A M et al, 1998, J. Exp. Med. 188:1413-9; Harrison, J K et al, 2001, J. Biol. Chem. 276:21632-41; Hughes P M et al, 2002, Glia 37:314-27; Hulshof S et al, 2003, J. Neuropathol. Exp. Neurol. 62:899-907; Imai T et al, 1997, Cell 91:521-30; Jung S et al, 2000, Mol. Cell. Biol. 20:4106-14; Lesnik P et al, 2003, J. Clin. Invest. 111:333-40; Meucci O et al, 2000, Proc. Natl. Acad. Sci. 97:8075-80; Mizuno T et al, 2003, Brain Res. 979:65-70; Nanki T et al, 2004, J. Immunol. 173:7010-6; Nishiyori A et al, 1998, FEBS Lett. 429:167-72; Rancan M et al, 2004, J. Cereb. Blood Flow Metab. 24:1110-8; Tarozzo G et al, 2002, Eur. J. Neurosci. 15:1663-8; Verge G M et al, 2004, Eur. J. Neurosci. 20:1150-60; Suzuki F et al, 2005 J. Immunol. 175:6987-96; Nanki T et al, 2004, J. Immunol. 173:7010-6; Harcourt J L et al, 2004 J. Infect. Dis. 190:1936-40; Ahn S Y et al, 2004 Am. J. Pathol. 164:1663-72; Yamashita K et al, 2003, Tohoku J. Exp. Med. 200:187-94; Fahy O L et al, 2003, Lab. Invest. 83:721-30; Hundhausen C et al, 2003, Blood 102:1186-95; Imaizumi T et al, 2002, Immunol. Cell Biol. 80:531-6; Holdsworth S R et al, 2000, Curr. Opin. Nephrol. Hypertens. 9:505-11; Chapman G A et al, 2000, J. Neurosci. 20:RC87; Chen S et al, 1998, J. Exp. Med 188:193-8.

In some embodiments, an antagonist of CX3CR1 is identified by monitoring the release of an enzyme upon degranulation or exocytosis by a cell capable of this function. For example, cells expressing CX3CR1 can be maintained in a suitable medium under suitable conditions and degranulation can be induced. In some embodiments cells are contacted with an agent to be tested, and enzyme release is assessed. The release of an enzyme into the medium can be detected and/or measured using a suitable assay, such as an immunological assay, or biochemical assay for enzyme activity. For example, the medium can be assayed directly, by introducing components of the assay (e.g., substrate, co-factors, antibody) into the medium before, simultaneous with, or after the cells and agent(s) are combined. The assay could also be performed on medium that had been separated from the cells or further processed (e.g., fractionated) prior to the assay. For example, convenient assays for monitoring the activity of an antagonist of CX3CR1 function can be performed by monitoring cellular responses induced by the active receptor, using suitable cells expressing the receptor. For instance, exocytosis (e.g., degranulation of cells leading to release of one or more enzymes or other granule components, such as esterases (e.g., serine esterases), perforin, and/or granzymes), inflammatory mediator release (such as release of bioactive lipids such as leukotrienes (e.g., leukotriene C₄)), and respiratory burst, can be monitored by methods known in the art or other suitable methods (see e.g., Taub, D. D. et al., 3. Immunol., 155: 3877-3888 (1995), regarding assays for release of granule-derived serine esterases; Loetscher et al., J. Immunol., 156: 322-327 (1996), regarding assays for enzyme and granzyme release; Rot, A. et al., J. Exp. Med., 176: 1489-1495 (1992) regarding respiratory burst; Bischoff, S. C. et al, Eur. J Immunol., 23: 761-767 (1993) and

Baggliolini, M. and C. A. Dahinden, Immunology Today, 15: 127-133 (1994); all of which are incorporated herein by reference).

In a preferred embodiment, an antagonist of CX3CR1 function does not significantly inhibit the function of other chemokine receptors (e.g., CCR1, CCR2, CXCR1,CCR3). Such CX3CR1-specific antagonists can be identified by suitable methods, such as by suitable modification of the methods described herein. For example, cells which do not express CX3CR1 but do express one or more other chemokine receptors (e.g., CCR2, CXCR1, CCR3) can be created or identified using suitable methods. Such cells or cellular fractions (e.g., membranes) obtained from such cells can be used in a suitable binding assay. For example, if a cell lacks CX3CR1 and contains only CCR3, the CX3CR1 antagonist can be assayed for the capacity to inhibit the binding of a suitable CCR3 ligand (e.g., RANTES, MCP-3) to the cell or cellular fraction, as described herein.

In some embodiments, the antagonist of CX3CR1 function is an agent that binds to CX3CR1. Such CX3CR1-binding antagonists can be identified by suitable methods, for example, in binding assays employing a labeled (e.g., fluorescent, luminescent, colorimetric, radioactive) antagonist. In another embodiment, the antagonist of CX3CR1 function is an agent that inhibits the binding of a CX3CR1 ligand to CX3CR1. In a preferred embodiment, the antagonist of CX3CR1 function is an agent that binds to CX3CR1 and thereby inhibit the binding of a CX3CR1 ligand to CX3CR1. Preferably, the antagonist of CX3CR1 function is, for example, a small organic molecule, natural product, protein (e.g., antibody, chemokine, cytokine), peptide or peptidomimetic. Several types of molecules that can be used to antagonize one or more functions of chemokine receptors are known in the art, including small organic molecules, proteins, such as antibodies (e.g., polyclonal sera, monoclonal, chimeric, humanized) and antigen-binding fragments thereof (e.g., Fab, Fab′, F(ab′).sub.2, Fv); chemokine mutants and analogues, for example, vMIP-II (Chen, S. et al., J. Exp. Med., 188:193-198 (1998)) and variants and analogues of vMW-II (see e.g., Wang et al., Protein Pept Lett. 13:499-501 (2006)), those disclosed in U.S. Pat. No. 5,739,103 issued to Rollins et al, WO 96/38559 by Dana Farber Cancer Institute and

WO 98/06751 by Research Corporation Technologies, Inc.; and peptides, for example, those disclosed in WO 98/09642 by The United States of America. The teachings of the above cited references are incorporated by reference herein in their entireties.

One some embodiments the agent is an antagonist of expression levels of a CX3CR1 ligand. A preferred embodiment contemplates the use of a nucleic acid, such as a small interfering RNA (siRNA), which blocks translation of a CX3CR1 ligand mRNA thereby causing a decrease in the expression level of the CX3CR1 ligand. In a preferred embodiment, the antagonist of a CX3CR1 ligand function clods not significantly inhibit the function of other chemokines (e.g., RANTES, MIP-1α, MCP-1). Such CX3CR1 ligand specific antagonists can be identified by suitable methods, such as by suitable modification of the methods described herein. For example, cells that do not express the receptor for a CX3CR1 ligand, but do express a receptor of another chemokine (e.g., CCRI which binds RANTES), can be identified. The CX3CR1 ligand antagonist can be assayed for the capacity to inhibit binding of a suitable ligand to such cells or cellular fractions (e.g., membranes) obtained from such cells.

In some embodiments, the antagonist of a CX3CR1 ligand function is an agent that binds the CX3CR1 ligand (e.g., a transmembrane CX3CR1 ligand such as fractalkine, a soluble CX3CR1 ligand such as soluble fractalkine). Such CX3CR1 ligand binding antagonists can be identified by suitable methods, for example, in binding assays employing a labeled (e.g., fluorescent, luminescent, colorimetric, radioactive) antagonist. For example, an antagonist of fractalkine function can be identified using a receptor binding assay with a labeled fractalkine (e.g., fluorescent, luminescent, colorimetric, radioactive label) that demonstrates the antagonist ability to inhibit the binding of fractalkine to the CX3CR1 receptor. In another example, the capacity of an agent to inhibit fractalkine-induced cellular adhesion is assessed. Cellular adherence can be monitored by methods known in the art or other suitable methods. In one embodiment, an agent to be tested can be combined with (a) non adherent cells which express mammalian fractalkine (e.g., the integral membrane form of fractalkine) or a functional variant thereof, and (b) a composition comprising a fractalkine receptor (e.g., a substrate such as a culture well coated with CX3CR1, a culture well containing adherent cells which express CX3CR1), and maintained under conditions suitable for fractalkine-receptor mediated adhesion. Labeling of cells with a fluorescent dye provides a convenient means of detecting adherent cells. Non-adherent cells can be removed (e.g., by washing) and the number of adherent cells determined. A reduction in the number of adherent cells in wells containing a test agent in comparison to suitable control wells (e.g., wells that do not contain a test agent) indicates that the agent is an antagonist of fractalkine function. The antagonist of fractalkine function can inhibit the function of transmembrane fractalkine, soluble fractalkine or other active fragments of fractalkine (e.g., fragments having chemoattractant activity).

In another embodiment, the antagonist of a CX3CR1 ligand function is an agent that binds to a mammalian CX3CR1 ligand and thereby inhibit the binding of the mammalian CX3CR1 ligand (e.g., a transmembrane CX3CR1 ligand such as fractalkine, a soluble CX3CR1 ligand such as soluble fractalkine) to a mammalian a CX3CR1 ligand receptor (e.g., human CX3CR1). Preferably, the antagonist of CX3CR1 ligand function is a compound that is, for example, a small organic molecule, natural product, protein (e.g., antibody, chemokine, cytokine), peptide or peptidomimetic. Antagonists of CX3CR1 ligand function can be prepared and/or identified using suitable methods, such as the methods described herein or suitable modifications thereof. For example, antibodies (e.g., polyclonal antibodies, monoclonal antibodies, antigen-binding fragments of antibodies) having binding specificity for a CX3CR1 ligand can be prepared by immunizing a suitable host with a CX3CR1 ligand (e.g., isolated and/or recombinant CX3CR1 ligand or a portion thereof) or with cells that express a CX3CR1 ligand. In one embodiment, the antagonist of CX3CR1 ligand function is an antibody or antigen-binding fragment thereof having binding specificity for a mammalian a CX3CR1 ligand (e.g., human fractalkine).

In a preferred embodiment, the present invention provides monoclonal antibodies that specifically bind to an isolated polypeptide comprised of at least five amino acid residues of a CX3CR1 or a CX3C ligand. These antibodies find use in the diagnostic and therapeutic methods described herein. In other embodiments, commercially available antibodies are utilized (e.g., available from any suitable source including, but not limited to, R & D System, Minneapolis, Min.).

The present invention contemplates the use of both monoclonal and polyclonal antibodies, as long as they can recognize and bind the protein of interest. Antibodies can be produced by using a protein of the present invention as the antigen according to a conventional antibody or antiserum preparation process. The protein used herein as the immunogen is not limited to any particular type of immunogen. For example, fragments (e.g., fragments may or may not retain native protein activity) of the protein may be used.

Fragments may be obtained by any methods including, but not limited to expressing a fragment of the gene, enzymatic processing of the protein, chemical synthesis, and the like.

Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol

(PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a CXCR3 or CCL ligand). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a CXCR3 or CCL ligand) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared, and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide-activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

In a more preferred embodiment, the present invention provides humanized antibodies that specifically bind to an isolated polypeptide comprised of at least five amino acid residues of a CX3CR1 or a known CX3C ligand. Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity for a CX3CR1 or a known CX3C ligand can be selected (e.g., from a phage library; see e.g., Krebber et al., U.S. Pat. No. 5,514,548; Hoogenboom et al., WO 93/06213, published Apr. 1, 1993). For example, nucleic acids encoding a chimeric or humanized chain can be expressed to produce a contiguous protein (for example, see Cabilly et al., U.S. Pat. No. 4,816,567; Cabilly et al, European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988); all references are incorporated herein in their entirety).

As described herein, there is a correlation between the function of CX3CR1 and/or the CX3C chemokine fractalkine and the course of neuroinflammatory disease. It is also demonstrated that antagonists of CX3CR1 prevent neuronal death in wild-type mice in two widely accepted animal models of human neuroinflammatory disorders.

The following examples are provided in order to demonstrate and further illustrate certain preferred embodiments and aspects of the present invention and are not to be construed as limiting the scope thereof.

### EXAMPLE 1

### Response of CX3CR1 Knock-Out Mice to MPTP Induced Neuronal Injury

To determine if CX3CR1 and/or a CX3C ligand (e.g. fractalkine) function was involved in the initiation, progression and/or maintenance of a neuroinflammatory disease, a study analyzing the function of CX3CR1 in mice treated with 1-methyl-4-phenyl-1,2,3,6-tetrahydropyridine (MPTP), a widely used model of Parkinson's disease was undertaken. The MPTP model used consisted of injecting knock-out mice intraperitoneally with 25 mg/kg of MPTP at two hour intervals for a total of four times. Following the series of injections, the mice were sacrificed at intervals of 2, 7,14, and 28 days. At the time of sacrifice, the animals were perfused transcardially with 4% paraformaldehyde and their brains dissected out and fixed in 4% paraformaldehyde overnight. The brains were subsequently cryoprotected with 30% sucrose over 2 days, at which point the midbrain and striatum areas were cut out and frozen on dry ice. Frozen sections (25 μm) of midbrain and striatum were obtained using a freezing cryostat. Brain tissue sections were immunostained with antibodies against tyrosine hydroxylase (TH) thereby labeling dopaminergic neurons in the midbrain. A fluorescent secondary antibody was used to further fluorescently label the cells. Images of the whole substantia nigra pars compacta nucleus were obtained using a

Zeiss Axiscope microscope (5× and 20×). Cell numbers were quantified using imaging software (NIH image).

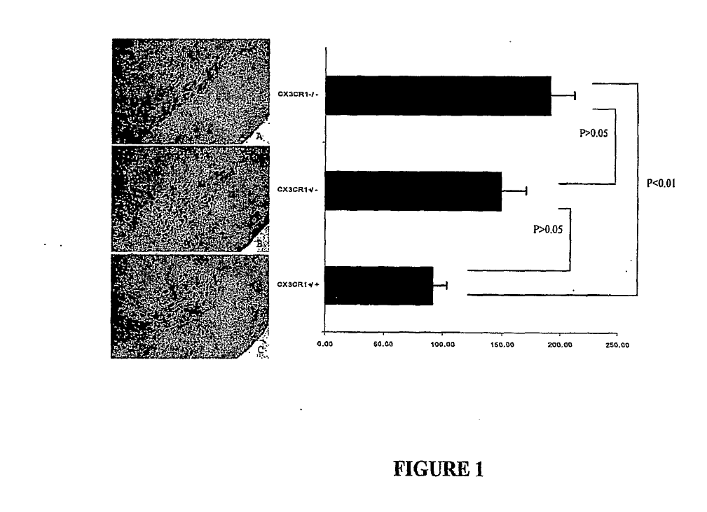

A large reduction in the amount of neuronal death in the substantia nigra pars compacta (SNpc) following treatment with MPTP was seen in knock-out mice. Results indicate a greater than 90% resistance to neuronal death in the homozygous CX3CR1 knock-out mice (−/−) when compared to heterozygous (+/−) and wild-type (+/+) controls (FIG. 1).

### EXAMPLE 2

Affect of Neutralizing Antibodies Against CX3CR1 on Neuronal Injury upon Challenge with MPTP

The ability of two commercially available neutralizing antibodies to CX3CR1, anti-rat CX3CR1 (Torrey Pines Biolabs, Inc., rabbit polyclonal, Catalog No. TP-501) and anti-human CX3CR1 (Torrey Pines Biolabs, Inc., rabbit polyclonal, Catalog No. TP-502) to prevent cell injury and death were tested. Wild-type C57B6 mice were given the same MPTP treatment as described in Example 1. The antibodies were injected into the lateral ventricle of the mice using a stereotaxic apparatus and a Hamilton syringe. Antibody injection was done 24 hours following MPTP treatment. A second control group received an injection of non-specific immunoglobulins. Mice were anaesthetized and a burr hole was drilled through the cranium with a microsurgical drill according to stereotaxic coordinates (Atlas of Paxinos and Paxinos). A Hamilton syringe was used to introduce either 2 μl of test antibody (1:100) or control immunoglobulin (1:100) into the lateral ventricle. The syringe was removed and the scalp was closed with a 6-0 nylon suture. The mice were allowed to recover and then sacrificed at days 2, 7,14, and 28 days following injection. A ˜50% reduction in neuronal death was observed upon staining with tyrosine hydroxylase and Nissl stains of the substantia nigra pars compacta area when the test antibody group was compared with the control group at 28 days after MPTP administration.

### EXAMPLE 3

### CX3CR1 Knockout in a Transgenic Alzheimer's Disease Mouse Model

CX3CR1 −/− mice were bred with a mouse model of Alzheimer's Disease (AD) (Mo/Hu APPswe PS18E9, Jackson Labs). The AD mice develop abundant amyloid plaques by 5-8 months of age. Amyloid plaque load in the cortex and hippocampus of the crossbred mice was compared by evaluating the effect of CX3CR1 knock out with the wild type CX3CR1 genotype found in the AD mice. Mice at six months of age were sacrificed and fixed by perfusion with 4% paraformaldehyde. The brains of age and sex matched mice were dissected out and fixed for 24 h. Brains were cryoprotebted with 30% sucrose, frozen on dry ice, and cut in 25 μm thick sections with a cryostat. To highlight amyloid plaques, sections were stained with either thioflavin S or antibody 4G8 raised against β-amyloid (Signet Laboratories). Sagittal and coronal brain sections were imaged using a Zeiss

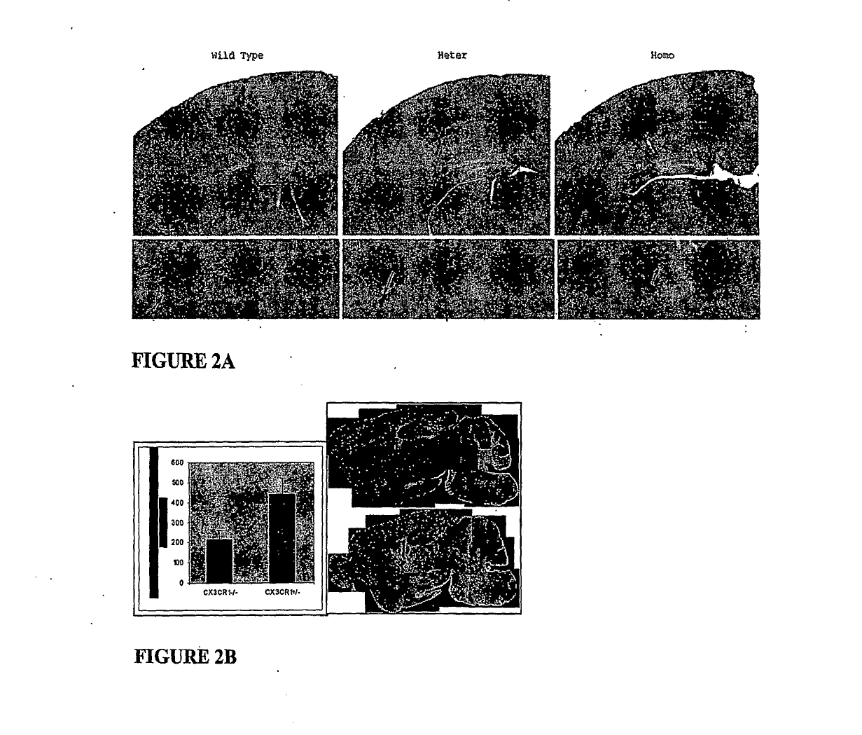

Axiovert microscope. Imaging software (NIH) was used to quantify and compare the number of amyloid plaques. Elimination of the CX3CR1 receptor in mice crossbred with an Alzheimer's mouse shows a greater than 50% reduction in amyloid plaque load in the cerebral cortex, hippocampus and basal ganglia when compared with wild type littermates at age 6 months (FIGS. 2a & b).

### EXAMPLE 4

Assessment of CX3CR1 Antagonists using a Radioactive Binding Assay

The capacity of an agent to antagonize CX3CR1 function is determined using a suitable functional assay, thereby assessing the usefulness of a particular antagonist as a potential therapeutic for neuroinflammatory diseases.

For example, membranes are prepared from cells which express CX3CR1, such as THP-1 cells (Raport, C. J. et al, Gene, 163:295-299 (1995)(American Type Culture Collection, Accession No. TIB202). Cells are harvested by centrifugation, washed twice with PBS (phosphate-buffered saline), and the resulting cell pellets frozen at −70° to −85° C. The frozen pellet are thawed in cold lysis buffer consisting of 5 mM HEPES (N-2-hydroxyethylpipera-zine-N′-2-ethane-sulfonic acid) pH 7.5, 2 mM EDTA (ethylenediaminetetraacetic acid), 5 μ/ml each of the protease inhibitors aprotinin, leupeptin, and chymostatin, and 100 μg/ml PMSF, at a concentration of 1-5×10⁷ cells/ml, to achieve cell lysis. The resulting suspension is mixed well to resuspend all of the frozen cell pellet. Nuclei and cell debris are removed by centrifugation of 400×g for 10 minutes at 4° C. The resulting supernatant is transferred to a fresh tube and the membrane fragments collected by centrifugation at 25,000×g for 30 minutes at 4° C. The resulting supernatant is aspirated and the pellet resuspended in a buffer consisting of 10 mM HEPES pH 7.5, 300mM sucrose, 1 μg/ml each aprotinin, leupeptin, and chymostatin, and 10 μg/ml PMSF (approximately 0.1 ml per each 10⁸ cells). All clumps are resolved using a mini-homogenizer, and the total protein concentration determined by suitable methods (e.g., Bradford assay, Lowry assay). The membrane solution is divided into aliquots and frozen at −70 to −85° C. until needed.

The membrane solution is used in a suitable binding assay. For example, membrane protein (2 to 20 μg total membrane protein)is incubated with 0.1 to 0.2 nM¹²⁵I-fractalkine with or without unlabeled competitor (e.g., fractalkine, vMIP-II (Chen, S. et al., J. Exp. Med., 188:193-198 (1998)) or various concentrations of compounds to be tested. ¹²⁵I-fractalkine is prepared by suitable methods. The binding reactions are performed in 60 to 100 μl of a binding buffer consisting of 10 mM HEPES pH 7.2, 1 mM CaCl₂, 5 mM MgCl₂, and 0.5% BSA, for 60 min at room temperature. The binding reactions are terminated by harvesting the membranes by rapid filtration through glass fiber filters (e.g., GF/B or GF/C, Packard) presoaked in 0.3% polyethyleneimine. The filters are rinsed with approximately 600 μl of binding buffer containing 0.5 M NaCl, dried, and the amount of bound radioactivity is determined by scintillation counting.

The CX3CR1 activity of the test antagonist is reported as the inhibitor concentration required for 50% inhibition (IC₅₀ values) of specific binding in receptor binding assays (e.g., using ¹²⁵I-fractalkine as ligand and THP-1 cell membranes). Specific binding is preferably defined as the total binding (e.g., total cpm on filters) minus the non-specific binding. Non-specific binding is defined as the amount of cpm still detected in the presence of excess unlabeled competitor (e.g., fractalkine). If desired, membranes prepared from cells that express recombinant CX3CR1 are used in the described assay.

### EXAMPLE 5

Assessment of CX3CR1 Antagonists using a Chemotaxis Assay

The capacity of an agent to antagonize CX3CR1 function can be determined in a leukocyte chemotaxis assay using suitable cells. Suitable cells include, for example, cell lines, recombinant cells or isolated cells that express CX3CR1 and undergo CX3CR1 ligand-induced (e.g., fractalkine-induced) chemotaxis. In one example, CX3CR1-expressing recombinant Li2 cells (see Campbell, et a. J. Cell Biol, 134:255-266 (1996)), peripheral blood mononuclear cells, or THP-1 cells, are used in a modification of a transendothelial migration assay (Carr, M. W., et al. T. A., Proc. Natl Acad Sci, USA, (91):3652 (1994)). Peripheral blood mononuclear cells are isolated from whole blood by suitable methods, for example, density gradient centrifugation and positive or preferably negative selection with specific antibodies. The endothelial cells used in this assay are preferably the endothelial cell line ECV 304, obtained from the European Collection of Animal Cell Cultures (Porton Down, Salisbury, U.K.). Endothelial cells are cultured onto 6.5 mm diameter Transwell culture inserts (Costar Corp., Cambridge, Mass.) with 3.0 μm pore size. Culture medium for the ECV 304 cells consists of M199+10% FCS, L-glutamine, and antibiotics. The assay media consists of equal parts RPMI 1640 and M199 with 0.5% BSA. Two hours before the assay, 2×10⁵ ECV 304 cells are plated onto each insert of the 24-well Transwell culture inserts and incubated at 37° C. Chemotactic factors such as the CX3C ligand fractalkine (diluted in assay medium) are added to the wells of a 24-well tissue culture plate in a final volume of 600 μL. Fractalkine is commercially available from, for example, Research Diagnostics Inc., Flanders, N.J. Endothelial-coated Transwells are inserted into each well and 10⁶ cellsof the leukocyte cells being studied are added to the top chamber of the tissue culture plate in a final volume of 100 μL of assay medium. The plate is incubated at 37° C. in 5% CO₂ for 1-2 hours. The leukocyte cells that migrate to the bottom chamber during incubation are counted, for example using flow cytometry.

To count cells by flow cytometry, 500 μL of the cell suspension from the lower chamber are placed in a tube and relative counts obtained for a set period of time, for example, 30 seconds. This counting method is highly reproducible and allows gating on the leukocytes and the exclusion of debris or other cell types from the analysis. Alternatively, cells are counted with a microscope. Assays to evaluate chemotaxis inhibitors are performed in the same way as the control experiment described above, except that antagonist solutions, in assay media containing up to 1% of DMSO co-solvent, are added to both the top and bottom chambers prior to addition of the cells. Antagonist potency is determined by comparing the number of cells that migrate to the bottom chamber in wells that contain antagonist, to the number of cells that migrate to the bottom chamber in control wells. Control wells may contain equivalent amounts of DMSO, but no antagonist.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method comprising: administering an inhibitor of CX3CR1 or a CX3C ligand to a subject having a neurological condition.

2. The method of claim 1, wherein said neurological condition is Alzheimer's disease.

3. The method of claim 2, wherein said inhibitor is administered under conditions such that amyloid plaque load in the neurological tissue of the subject is reduced compared to a subject not administered said inhibitor.

4. The method of claim 3, wherein said neurological tissue is one or more of cerebral cortex, hippocampus, and basal ganglia.

5. The method of claim 1, wherein said inhibitor comprises an immunoglobulin that binds to said CX3CR1 or said CX3C ligand.

6. The method of claim 1, wherein said inhibitor comprises a chemokine mutant that does not activate CX3CR1.

7. The method of claim 1, wherein said inhibitor comprises a chemokine analogue that does not activate CX3CR1.

8. The method of claim 1, wherein said inhibitor comprises a chemokine fragment that does not activate CX3CR1.

9. The method of claim 1, wherein said inhibitor comprises

10. A method for identifying inhibitors of CX3CR1 or a CX3C ligand comprising:
a) exposing a sample to a candidate compound; and
b) detecting the activity of CX3CR1 or a CX3C ligand associated with a neurological disorder upon exposing said sample to said candidate compound.

11. The method of claim 10, wherein said neurological condition is Alzheimer's disease.

12. The method of claim 11, wherein said detecting comprises detection of plaque.

13. The methods of claim 10, wherein said candidate compound is a peptide, an antibody, or a small molecule.

14. A method of claim 10, wherein said candidate compound is an siRNA.
